(19) United States
(12) Patent Application Publication
(10) Pub. No.: US 2013/0315820 A1
(43) Pub. Date: Nov. 28, 2013

(54) USE OF AN ANTIBODY DIRECTED AGAINST A MEMBRANE PROTEIN

(71) Applicants: Nathalie Fournier, Erquinghem-Lys (FR); Christophe Romeuf, Lambersart (FR)

(72) Inventors: Nathalie Fournier, Erquinghem-Lys (FR); Christophe Romeuf, Lambersart (FR)

(73) Assignee: LFB BIOTECHNOLOGIES, Courtaboeuf Cedex (FR)

(21) Appl. No.: 13/989,572

(22) PCT Filed: Dec. 12, 2011
(86) PCT No.: PCT/FR2011/052949

§ 371 Date: Aug. 2, 2013

(30) Foreign Application Priority Data
Dec. 13, 2010 (FR) 10/60428

(51) Int. Cl. C07K 16/30; A61K 45/06; A61K 39/395

(52) U.S. Cl. CPC C07K 16/3061; A61K 39/39558; A61K 45/06
USPC 424/1.49; 424/156.1; 424/174.1; 424/133.1; 424/142.1; 424/178.1; 424/183.1; 424/135.1

(57) ABSTRACT

An antibody directed against the BDCA-2 protein for the prevention or the treatment of the pathologies involving activation of the plasmacytoid dendritic cells.

A subject of the present invention is the use of an antibody directed against a membrane protein, and in particular for the treatment of pathologies.

The antibodies used in clinical medicine, with the aim of controlling an auto-immune or inflammatory pathology, are to date mainly the anti-CD20 antibodies and in particular anti-CD20 Rituxan. The problem associated with this type of treatment is due to the fact that the anti-CD20 will eliminate all of the pre-B and mature B lymphocyte cells, but also approximately 20% of the plasmocytes and a population of memory B lymphocytes (Gonzales-Stawinski Clin Immunol 2001, 98, 175; Looney, Ann rheum dis 2002, 61, 863) thus leading to an immuno-deficient state associated with a hypogammaglobulinaemia.

A need therefore exists to find an alternative to this B depletion by reducing the number of plasmacytoid dendritic cells, cells which are key to the initiation of the pathological immune response, and therefore attenuate the immune response of the patient and consequently of the auto-reactive B cells.

The dendritic cells (DCs) are cells of the antigen presenting immune system (APCs) forming part of the reticulohistiocytosis system. Under certain conditions, the DC cells have cytoplasmic projections similar to the dendrites of neurons.

The dendritic cells have two main functions:
triggering the adaptive immune response, in which the main role is played by the T lymphocytes and the B lymphocytes, directed against the “non-self' antigens
maintaining the central tolerance to “self' in the thymus, by the process involving the T lymphocytes called negative selection.

Numerous sub-populations of DC cells have been characterized in mice, without however being correspondingly identified in humans.

The DCs are capable of being differentiated into various sub-types, according to the stimuli that they receive. Two major types of DC cells exist: conventional DC cells, plasmacytoid DC cells and inflammatory DC cells.

Conventional DC cells comprise:
migratory DC cells which reside, in the basal state, in the peripheral zone. Following the phagocytosis of an antigenic particle and/or the reception of signals, they migrate towards the secondary lymphoid ganglions via the lymphatic vessels; they arrive in the mature state in the lymphoid organs where they present the antigen to the naive T lymphocytes; for example Langerhans cells or also the D cells of the mucous membranes can be mentioned;
the lymphoid organ-resident immature DC cells; these cells collect and present the self or foreign antigens even within the lymphoid organs; they comprise the majority of the DC cells of the thymus of the spleen.

The inflammatory DC cells are recruited into the tissues following an inflammation or an infection but are never presented without any stimulation. It is currently supposed that the inflammatory DC cells would principally originate from the differentiation of blood monocytes.

The plasmacytoid DC cells differ from conventional DC cells in that they are circulating, round and without dendrites in the basal state, although they finish by differentiating to conventional DCs after activation. They are therefore capable of presenting the antigen. After stimulation, in general by a viral antigen, they produce a large quantity of class I interferons (IFN). These cells are essentially involved in the anti-viral response and in auto-immune disorders.

The plasmacytoid DC cells (also called plasmacytoid DC cells) have been characterized phenotypically: they express the CD4 and BDCA-2 and 4 markers. Their phenotype is therefore CD4+, CD11c−, Lin−, BDCA-2+, BDCA-4+.

The plasmacytoid DC cells can be at the origin of haematopoietic tumours in which they acquire an additional marker which is CD56+. This is why CD4+/CD56+ haematopoietic tumours are mentioned (BPDCN: Blastic plasmacytoid dendritic cells neoplasm).

The CD4+/CD56+ haematopoietic tumours are rare haematological neoplasms (1% of acute leukaemias), which present in the form of cutaneous nodules associated with a lympho-adenopathy or a swelling of the spleen and with a frequent cytopaenia. These cutaneous manifestations are very rapidly followed by an infiltration of the bone marrow. Due to the expression of the CD56+ marker, these pathologies were first classified as NK cell lymphomas. But the absence of myeloid, T or B or NK lymphoid line markers has given rise to complementary studies which have made it possible to refine the classification so as to finally show that the so-called type II dendritic cells or plasmacytoid DC cells are at the origin of CD4+/CD56+ haematopoietic tumours [Chaperot L et al., 2001, Blood. 2001 May 15; 97(10):3210-7, Herling M, Jones D., 2007, Am J Clin Pathol. 2007 May; 127(5):687-700]

Current treatment of these pathologies is based on chemotherapy which allows a complete remission for approximately 2 patients out of three but where relapses are frequent and occur soon (approximately 9 months). The overall survival median is approximately 13 months. Another alternative treatment is the allograft of haematopoietic cells which nevertheless also does not lead to long-term survival.

Consequently, a real need currently exists to find a treatment for these CD4+/CD56+ pathologies.

Means of treating these pathologies involving cells expressing markers of the dendritic cells are proposed in the prior art.

In particular, molecules targeting the BDCA differentiation markers have been proposed.

For example patent EP 1 301 539 can be mentioned which describes a polypeptide with a particular sequence specifically binding to the BDCA-2 protein and a pharmaceutical composition comprising it.

European patent application EP 1 783 141 discloses a composition making it possible to induce an immune response in a patient, said composition comprising dendritic cells treated beforehand with an anti-BDCA-2 antibody and expressing an antigen (tumorous, viral, bacterial etc.). This consists of a treatment by cell therapy.

Application WO 01/365487 describes monoclonal antibodies directed against the BDCA-2 protein, and in particular the clones AC144, AD5-13A11 and AD5-4B8, as well as derived fragments. Moreover, this application describes the use of monoclonal antibodies for the treatment of pathologies such as viral infections, auto-immune diseases and tumours.

Application WO 2006/037247 describes the use of monoclonal antibodies directed against the BDCA-2 protein, in the context of the treatment of a particular auto-immune disease: psoriasis.

Nestle et al., (Nestle et al. 2005, J. Exp. Med, vol 202, No. 1, pp: 135-143) teaches that antibodies directed against the BDCA-2 protein can be injected by intravenous route, for a therapeutic purpose in the context of the treatment of psoriasis.

Blomberg et al., (Blomberg et al., 2003, Arthritis and Rheumatism, vol 48, No. 9, pp: 2524-2532) teaches the use of antibodies directed against the BDCA-2 protein, said vaccines being used for an auto-immune disease: Lupus Erythematosus. The authors show that said antibodies are capable of inhibiting the production of interferon α (IFN-α). Application US 2010/1896641 describes antibodies directed against the BDCA-2 protein in the context of the treatment of tumours.

Therefore it should be noted that the use of antibodies directed against the BDCA-2 protein is widely described in the prior art.

Nevertheless, the prior art is silent as regards the treatment of pathologies involving CD4+/CD56+ cells.

However, to date no specific and effective treatment exists which allows the treatment or the prevention of pathologies involving plasmacytoid dendritic cells, and in particular CD4+/CD56+ tumours.

Therefore a subject of the invention is to overcome the drawbacks of the prior art, and in particular to provide alternative treatments.

Moreover, a purpose of the invention is to provide a novel treatment that is effective and specific to CD4+/CD56+ cell tumours.

A subject of the invention is also medicinal compositions for treating and/or preventing said pathologies.

The invention relates to a monoclonal or polyclonal antibody directed against the BDCA-2 protein for its use in the context of the prevention or treatment of pathologies involving activation of the plasmacytoid dendritic cells.

The invention is based on the unexpected observation made by the Inventors of the effect of anti-BDCA-2 antibodies on the progression of tumours, in particular haematopoietic tumours.

The invention is distinguished from the prior art in that it involves the destruction of the plasmacytoid DC cells by apoptosis or by the ADCC mechanism.

The use of anti-IFN-α antibodies in the treatment of patients suffering from auto-immune and inflammatory pathologies induces a systemic neutralization of the IFN-α, which potentially increases the risk of opportunistic infections. In fact this cytokine is produced in response to an infectious agent in particular a viral agent.

In order to respond to this problem, the object of the invention consists of eliminating the plasmacytoid DC cells, the main source of IFN-a, recruited at the level of the inflammatory lesions and responsible for the local inflammation, this being particularly well described in the case of psoriasis for example. The advantage of the invention therefore resides in elimination of the IFN-α preferentially at the site of the inflammation and not systemically, thereby reducing the risks linked to the immunodeficiency induced by the anti-IFN-αs.

A certain and additional advantage resides in the absence of effect on the leucocytes (Papot, 2002, rev med interne, 23, supp 4), the macrophages (Levesque M C, 1999, Arthritis Rheum, 42, 569) and the fibroblasts (Houglum, J E, 1893, 2.20) and thus preserves the sources of IFN-α produced by these cell types.

Another advantage consists of the reduction, via the elimination of the plasmacytoid DCs, of other pro-inflammatory cytokines such as IT-6 also secreted by the plasmacytoid DCs in an inflammatory environment and leading to a Th1 response for example.

Moreover, contrary to generally accepted concepts, the object of the invention consists of taking advantage of the hyper-activation of the immune system at the site of the inflammation, in particular the activation of the effector cells including the NK cells and the macrophages and due to the presence of numerous pro-inflammatory cytokines and chemokines in order to finally obtain a reduction in the inflammation. In fact, the authors have shown that the anti-BDCA-2 antibody cited has a better cytotoxic activity against the pDCs in the presence of molecules the levels of which are increased in auto-immune and inflammatory pathologies (i.e. IFN-γ, TNF-α, etc.).

The BDCA-2 protein is expressed specifically at the surface of the plasmacytoid DC cells, and is a type II protein belonging to the type C lectins.

In the context of the present invention, the term “antibody” refers to an immunoglobulin, a protein constituted by 4 chains participating in the acquired immune response or to a fragment of immunoglobulin.

Immunoglobulins are well known to a person skilled in the art and are constituted by an assembly of two dimers each constituted by a heavy chain and a light chain. The multimeric complex is assembled by the binding of a light chain and a heavy chain by means of a disulphide bridge between two cysteines, the two heavy chains being themselves also connected together by two disulphide bridges.

Each of the heavy chains and the light chains is constituted by a constant region and a variable region. The assembly of the chains which make up an antibody makes it possible to define a characteristic three-dimensional structure in the shape of a Y, where
the base of the Y corresponds to the constant region Fc which is recognized by the complement and the Fc receptors, and
the end of the arms of the Y correspond to the respective assembly of the variable region of the light chain and the variable region of the heavy chain which are recognized by a specific antigen.

More precisely, each light chain is constituted by a variable region (V_{L}) and of a constant region (CO. Each heavy chain is constituted by a variable region (V_{H}) and a constant region constituted by three constant domains C_{H1}, C_{H2} and C_{H3}. The domains C_{H2} and C_{H3} make up the domain Fc.

The antibodies described in the invention are isolated and purified, and are different from natural antibodies. These antibodies are mature, i.e. they have an ad hoc three-dimensional structure which allows them to recognize the antigen, and have all the post-translational modifications essential for their antigen recognition.

In one aspect of the invention, the antibodies are polyclonal.

In another aspect, they are monoclonal antibodies, i.e. they recognize only a single antigenic determinant in BDCA-2, in contrast to the polyclonal antibodies which correspond to a mixture of monoclonal antibodies, and can therefore recognize several antigenic determinants in the same protein.

The monoclonal antibodies of the invention can be obtained by techniques well known to a person skilled in the art, and in particular by the technique of cell fusion, or also the technique of cloning the sequences of the heavy and light chains, by the technique of phage or ribosome display, by immunization of mice having the human immunoglobulin repertoire and ad hoc expression in a cell or a transgenic animal.

These techniques are well known to a person skilled in the art.

By “plasmacytoid dendritic cells” is meant the sub-population of dendritic cells also called DC2 characterized by the HLA-DR+, CD11c−, CD123+ and CD45RA+ markers, present in the lymphoid organs and also in circulation in the blood and characterized by their ability to secrete type I IFN in the presence of a viral infection.

In the invention, by “activation of the plasmacytoid dendritic cells” is meant the different mechanisms having the effect of activating the determined proliferation and cytokine secretion, in particular class I interferons, and the phenotypical and morphological modification of the plasmacytoid cells.

In an advantageous embodiment, the invention relates to a monoclonal or polyclonal antibody directed against the BDCA-2 protein for its use as defined previously, where said pathologies involving plasmacytoid dendritic cells are chosen from tumours, auto-immune diseases and inflammatory diseases.

Advantageously, the invention relates to a monoclonal or polyclonal antibody directed against the BDCA-2 protein for its above-mentioned use, where the tumours are preferably haematopoietic tumours.

In the invention, by “haematopoietic tumours” is meant tumours or tumorous diseases involving cells of the blood line, or haematopoietic cells. These tumours can also be called malignant haemopathies.

The malignant haemopathies include
leukaemias: tumours where the blood cells proliferate abnormally in the blood,
lymphomas: tumours where the blood cells proliferate abnormally in the secondary lymphoid organs (ganglions, spleen etc.).
or Kahler's disease: tumours where the blood cells proliferate abnormally in the bone marrow.

Another advantageous embodiment of the invention relates to a monoclonal or polyclonal antibody directed against the BDCA-2 protein directed against the BDCA-2 protein for its use as defined previously, where the haematopoietic tumours are tumours of phenotype CD4+, CD56+.

By “CD4+/CD56+ haematopoietic tumours” is meant malignant haemopathies in which the cancer cells jointly express the two CD4 and CD56 markers. These diseases include cutaneous agranular CD4+/CD56+ lymphomas, agranular CD4+/CD56+ lymphoma cutis, blastic NK leukaemias/lymphomas, NK lymphomas [Ng A P et al. 2006. Haematologica 91 (1): 143-4; Kim Y et al. 2005 J. Korean Med. Sci. 20 (2): 319-24; Chan J K, et al. 1997 Blood 89 (12): 4501-13]

In another advantageous embodiment, the invention relates to a monoclonal or polyclonal antibody directed against the BDCA-2 protein directed against the BDCA-2 protein for its use as defined above, where the auto-immune and inflammatory diseases are chosen from diseases of the connective tissues, different types of sclerosis, pulmonary auto-immune inflammation, Guillain-Barre's syndrome, auto-immune thyroiditis, mellitis, myasthenia gravis, graft-versus-host disease, inflammatory auto-immune disease of the eye, psoriasis, Basedow's disease (hyperthyroidism), Hashimoto chronic thyroiditis (hypothyroidism), systemic lupus erythematosus (SLE), Goodpasture's syndrome, pemphigus, myasthenia, diabetes due to insulin resistance, auto-immune haemolytic anaemia, auto-immune thrombocytopaenic purpura, rheumatoid arthritis, scleroderma, polymyositis and dermatomyositis, Biermer's anaemia, Gougerot-Sjogren's disease, glomerulonephritis, Wegener's disease, Horton's disease, polyarteritis nodosa and Churg and Strauss syndrome, Still's disease, atrophic polychondritis, Behçet's disease, multiple sclerosis, spondylitis, Crohn's disease, monoclonal gammapathy, Wegener's granulomatosis, lupus, Horton's disease, Reiter's disease, hemorrhagic rectocolitis, psoriatic arthritis, sarcoidosis, ankylosing spondylitis, auto-immune bullous dermatitis, collagenous colitis, dermatitis herpetiformis, familial Mediterranean fever, glomerulonephritis with IgA deposits, membranous glomerulonephritis, auto-immune hepatitis, Lambert-Eaton myasthenic syndrome, sympathetic ophthalmia, bullous pemphigoid, pemphigus, idiopathic thrombopenic purpura, Fiessinger-Leroy-Reiter syndrome, auto-immune thyroiditis, uveo-meningoencephalitis and dermatitis herpetiformis.

In another preferred embodiment of the invention, the pathologies involving plasmacytoid dendritic cells are auto-immune diseases, preferably auto-immune diseases characterized by an increased secretion of type I IFN.

In an advantageous embodiment, the invention relates to a monoclonal or polyclonal antibody directed against the BDCA-2 protein directed against the BDCA-2 protein for its use according to the preceding definition, where the tumours are haematopoietic tumours are chosen from lymphoproliferative syndromes and myeloma (Waldenström's disease, chronic lymphoid leukaemia, multiple myeloma, primitive amyloidosis), lymphomas (follicular lymphoma, diffuse lymphoma, lymphoma of the marginal zone, Hodgkin's disease), aplasia and leukaemias and myelodysplasias (acute lymphoblastic leukaemia, acute myeloid leukaemia, myelodysplasia, medullar aplasia), myeloproliferative syndromes (myelofibrosis, polycythaemia vera, essential thrombocythaemia, chronic myeloid leukaemia).

In a yet more advantageous embodiment, the invention relates to a monoclonal or polyclonal antibody directed against the BDCA-2 protein directed against the BDCA-2 protein for its use according to the preceding definition, where said antibody is chosen from the following antibodies: a murine antibody, a chimeric antibody, a humanized antibody and a human antibody.

It is well known to a person skilled in the art that the murine antibodies are antibodies produced by mice cells and the human antibodies are produced by human cells.

However, in the invention the preceding definitions of human and murine antibodies will be generalized to any human or mouse antibody comprising the amino acid sequences of their heavy chains and their light chains. Also, a murine antibody is an antibody in which the sequences of the heavy chains and the light chains which constitute it are sequences the corresponding nucleic acid of which is found in the genome of the murine B cells. This antibody is therefore constituted by murine amino acid sequences, whatever the origin of the cell which allows its production.

For example, the mouse antibody sequences expressed in macaque cells will produce murine antibodies.

The above definition applies mutatis mutandis to human antibodies.

By “chimeric antibody” is meant in the invention an isolated antibody, in which the sequence of each light chain and/or of each heavy chain which constitutes it comprises or consists of a hybrid sequence originating from at least two distinct animals. In particular the chimeric antibodies of the invention are human/macaque or human/mouse hybrids, which signifies that a region of the sequence of the light chains and the heavy chains originates from the sequence of a macaque or mouse immunoglobulin, and that the remainder of the sequence of said heavy chains and said light chains originates from the sequence of one, or optionally several, human immunoglobulins.

By “humanized antibody” is meant in the invention an antibody originating from an animal other than a human in which the sequences of the heavy chains and the light chains other than the CDRs have been replaced by the corresponding sequences of one or more antibodies of human origin. The antibody is therefore in the main constituted by human sequences, but its specificity for the antigen conferred by the CDRs originates from another species.

In yet another advantageous embodiment, the invention relates to a monoclonal or polyclonal antibody directed against the BDCA-2 protein for its above-mentioned use, the tumours are solid tumours chosen from cancer of the lung, kidney, liver, pancreas, melanoma and ovary.

In yet another advantageous embodiment, the invention relates to a monoclonal or polyclonal antibody directed against the BDCA-2 protein for its above-mentioned use, where said antibody is a chimeric antibody and preferably a chimeric antibody chosen from a chimeric murine/human antibody or a chimeric human/macaque antibody.

In yet another advantageous embodiment, the invention relates to a monoclonal or polyclonal antibody directed against the BDCA-2 protein for its above-mentioned use, where said antibody is a humanized antibody.

In yet another advantageous embodiment, the invention relates to a monoclonal or polyclonal antibody directed against the BDCA-2 protein for its above-mentioned use, where said antibody is a human antibody.

In yet another advantageous embodiment, the invention relates to a monoclonal or polyclonal antibody directed against the protein directed against the BDCA-2 protein for its above-mentioned use, where said antibody has a fucosylation level of less than 60% of the glycosylated forms.

The antibody according to the invention has a low quantity of fucose on the glycan chains borne by said antibody. This quantity of fucose, or level of fucose, is defined as the average proportion of fucose borne by all the antibodies, with respect to the maximum quantity of fucose that the glycan chains can bear.

Another aspect of the invention relates to monoclonal or polyclonal antibody fragments directed against the BDCA-2 protein for its use as defined previously

The term “antibody fragment” defines a portion of antibody, preferably a binding site to the antigen or a variable region of the antibody. Examples of fragments include Fv, Fab, F(ab′)2, Fab′, dsFv, scFv, sc(Fv)2, diabody, triabody or tetrabody, and multispecific antibodies made up of different fragments. All of these antibody fragments are well known to a person skilled in the art.

For examples, a few definitions are given hereafter.

The term “Fab” denotes an antibody fragment with a molecular mass of approximately 50,000 daltons and having an antigen-binding activity. It comprises approximately half of the N-terminal side of the heavy chain and the entire light chain bound by a disulphide bridge. Fab can be obtained in particular by the treatment of IgG with a protease, papain.

The term “F(ab′)2” denotes a fragment of approximately 100,000 daltons and an antigen-binding activity, the connection by a disulphide bridge of two Fab [fragments] described above. It can be obtained by the treatment of IgG with a protease, pepsin.

A “scFv” (single chain Fv) is a VH:VL polypeptide synthetized by using the genes encoding the VL and VH domains and a sequence encoding a peptide intended to bind these domains. An scFv according to the invention includes the CDRs maintained in an appropriate conformation, for example by using genetic recombination techniques.

The dimers of ScFv correspond to two molecules of scFv bound together by a peptide bond.

ScFvs can also serve as base modules for the development of multimeric structures (dimeric: “diabody”, trimeric: “triabody”, tetrameric: “tetrabody”). Thus, the diabody is a dimer of scFv. The binding sequence is shorter, and therefore does not allow self-association between the heavy and light chains in a single scFv. This dimer fragment has the additional property of maintaining the double valency that the parent antibody possesses.

Also, in the invention, by “triabody” is meant the trivalent association of scFv, said triabody can bind to 3 identical or different antigens.

In the invention, a “tetrabody” corresponds to the tetravalent association of scFv, said tetrabody can bind to 4 identical or different antigens.

Another subject of the invention is an antibody or antibody fragment, where said antibody or antibody fragment is coupled to a bioactive molecule chosen from radio-isotopes, non-radioactive metals, toxins, nucleic acids, cytotoxic agents or also enzymes. Furthermore, the invention relates to a monoclonal antibody or monoclonal antibody fragment as defined previously, where said antibody or antibody fragment is coupled to a bioactive molecule chosen from radio-isotopes, non-radioactive metals, toxins (chosen from ricin, abrin, diphtheria toxin), the nucleic acids chosen from the anti-sense RNAs, cytotoxic agents chosen from mitomycin C, methotrexate, adriamycin, enzymes such as RNases, biotin, avidin or streptavidin.

Such antibodies coupled to a bioactive molecule are capable of specifically addressing a radio isotope, an enzyme, a heavy metal, or also a toxin, grafted to said antibody to a determined target cell, in this case plasmacytoid DC cells.

The following radio-isotopes At211, I131, I125, Y90, Re186, Re188, Sm153, Bi212, P32 can be mentioned as advantageous isotopes, this list not being limitative.

The toxins that can be grafted to the antibody according to the invention are chosen from ricin, tetanus toxin, abrin, diphtheria toxin.

The cytotoxic agents chosen from the antifolates (methotrexate, pemetrexed, raltitrexed) the anti-purines (cladribine, fludarabine, azathioprine, azathioprine, mercaptopurine, 5-fluorouracil capecitabine, cytarabine, gemcitabine), the inhibitors of topoisomerases I and II, the alkylating agents (chlormethine, cyclophosphamide, ifosfamide, carmustine, fotemustine) and related agents (mitomycin C, cisplatin, carboplatin, oxaliplatin), the intercalating agents, the anthracyclins (daunorubicin, doxorubicin and chlorydrate, epirubicin, idarubicin, bleomycin), the taxanes, the tyrosine-kinase specific inhibitors (imatinib, Erlotinib), can also be used. The mixture of these molecules or compounds that can be grafted on the antibody according to the invention are well known to person skilled in the art, and it is straightforward to determine the molecule to use.

The invention also relates to a combination product comprising an antibody or antibody fragment mentioned above, in combination with an anticancerous, agent for a use simultaneously, separately or spread over time, in tumorous therapy.

The antineoplastics which can be used in the invention are in particular chosen from
the anti-metabolics such as the antifolics (methotrexate, raltitrexed, and pemetrexed), the antipurines (mercaptopurine, thioguanine, pentostatin, cladribine and fludarabine), the antipyrimidines (5-fluorouracil, tegafur-uracil, capecitabine and cytarabine), and other anti-metabolics (hydroxycarbamide or hydroxyurea and gemcitabine),
the alkylating agents such as nitrogen mustards (chlorambucil, melphalan, chlormethine or metachloroethamine, estramustine, ifosfamide and cyclophosphamide), the nitroso-ureas (fotemustine, lomustine, carmustine and streptozocin), the platinum-based antineoplastics (carboplatin, cisplatin and oxaliplatin), the ethylene imines (thiotepa and altretamine), the triazenes (procarbazine, temozolomide and dacarbazine) and other alkylating agents (busulfan, mitomycin C and pipobroman),
   the intercalating agents such as the derivatives of the camptothecin (irinotecan and topotecan) the anthracyclines (epirubicin, daunorubicin, doxorubicin, pirarubicin and idarubicin) and other intercalating agents (mitoxantrone, amsacrine, elliptinium, actinomycin d or dactinomycin, etoposide and bleomycin), and
the molecules having an action on the mitotic spindle such as the vinca-alkaloids or spindle poisons (vinorelbine, vindesine, vincristine and vinblastine), the taxoids or stabilizers of the spindle (paclitaxel and docetaxel), the tyrosine kinase inhibitors (dasatinib, erlotinib, imatinib, sorafenib and sunitinib).

The above-mentioned molecules are given by way of indication but does not limit the scope of the invention. The above-mentioned compounds correspond to the international non-proprietary names (INN) and a person skilled in the art can very easily obtain the corresponding brand names from different suppliers.

In an advantageous embodiment, the invention relates to an above-mentioned combination product for a use in the treatment of haematopoietic tumours.

In an advantageous embodiment, the invention relates to an above-mentioned combination product, for its use in the treatment of tumours of phenotype CD4+, CD56+.

### FIGURE LEGENDS

FIG. 1 represents a histogram showing the depletion of BDCA-2+ plasmacytoid DC cellss in human PBMCs from a healthy donor in the presence of the anti-BDCA-2+ rabbit polyclonal antibody. The % of positive cells is obtained by a double ILT7/BDCA-2 labelling.

The black column represents the percentage of plasmacytoid DCs without treatment with the antibody (negative control 1), the white column represents the percentage of plasmacytoid DCs treated with a control antibody (which is not directed against BDCA-2; negative control 2) and the hatched column represents the percentage of plasmacytoid DCs treated with the anti BDCA-2 antibody.

FIG. 2 represents a histogram showing the amount of IFN-α after depletion of the plasmacytoid DCs with an anti-BDCA-2+ rabbit polyclonal antibody and activation by CpG.

The black column represents the percentage of IFN-α secretion by the total PBMCs, the white column represents the percentage of IFN-α secretion by the total PBMCs depleted of plasmacytoid DCs and the hatched column represents the percentage of IFN-α secretion by the plasmacytoid DCs alone.

FIG. 3 represents a histogram showing the ADCC activity of the anti-BDCA-2 antibody polyclonal (black bars) on the Jurkat-BDCA-2 cells. The results are expressed as a percentage of lysis of the Jurkat-BDCA-2 cell as a function of the quantity of antibodies added, expressed as a dilution factor of the initial antibody solution (A: 0, B: 1/250 and C: 1/25). Average +/−standard deviation. In the control, the lysis percentage is measured using a control antibody (white bars).

The Y-axis represents the lysis percentage.

### EXAMPLES

### Example 1

### Preparation of Cell Lines Expressing the BDCA-2 Protein

The complementary DNA of BDCA-2 is amplified by PCR and cloned in the expression vector pcDNA3.1(−) at restriction sites HindIII/BamHI.

The pcDNA vector containing BDCA-2 is transfected by nucleofection or Fugene HD on the U937 or THP-1 cell line.

48 hours after transfection, the expression of BDCA-2 by the transfected cells is monitored by flow cytometry, using an anti-BDCA-2 antibody and an antibody coupled to a fluorophore.

The BDCA-2+ cells are screened by positive selection using magnetic beads coupled to the anti-BDCA-2 antibody (Myltenyi kit).

The screening is repeated until 100% positive cells expressing BDCA-2 is obtained.

### Example 2

### Measurement of the Effect of the Anti-BDCA-2 Antibody According to the Invention

The effect of the anti-BDCA-2 antibody according to the invention is measured in mice or humans depending on its type.

A means of measuring the activity of the antibody is by measuring the dependent cell lysis of the antibody (ADCC) in the presence of killer cells (Natural Killer (NK) or T lines transfected with Fc receptors).

Measurement of the ADCC for a Murine Antibody or an Antibody having an Fc Fragment of Murine Origin

The killer cells (mouse effector cells or T lines transfected with murine FcRs) are incubated with transfected target BDCA-2 cells (Example 1) or plasmacytoid dendritic cells from leukaemic patients (CD4+/CD56+) or patients suffering from an inflammatory and/or auto-immune disease, in an E/T ratio of 15/1, in the presence of different concentrations of anti-BDCA-2 antibody.

After incubation for 4 or 16 hours, the cytotoxic activity induced by the anti-BDCA-2 antibody is measured by colorimetry by assaying the release of lactate dehydrogenase (LDH) (Roche Diagnostics—Cytotoxicity Detection Kit LDH ref 11644793001) in the supernatants.

In the case of a low intra-cell LDH level, the cells are labelled with a fluorescent agent and the lysis is estimated by measuring the quantity of fluorescence released in the supernatant.

The results of specific lysis are expressed as a percentage of lysis as a function of the antibody concentration. The EC₅₀ values (quantity of antibodies inducing 50% of the maximum lysis) as well as the Eₘₐₓ values (percentage of maximum lysis) are calculated using PRISM software.

ADCC Measurement for an Antibody having an Fc Fragment of Human Origin

Killer cells (PBMC, NK cells, or T cells transfected with human CD16) are purified beforehand by the negative depletion technique developed by the company Miltenyi (Miltenyi Biotec—NK cell isolation kit human ref 130-092-657), starting from peripheral blood from healthy donors. The ADCC technique consists of incubating the NK cells with the transfected target BDCA-2 cells or with plasmacytoid DC cells from leukaemic (CD4+/CD56+) patients or patients suffering from inflammatory and/or auto-immune disease, in an E/T ratio of 15/1, in the presence of different concentrations of anti-BDCA-2 antibody.

After incubation for 4 or 16 hours, the cytotoxic activity induced by the anti-BDCA-2 antibody is measured by colorimetry by assaying in the supernatants, an intracellular enzyme called lactate dehydrogenase (LDH) released by the lysed target cells (Roche Diagnostics—Cytotoxicity Detection Kit LDH ref 11644793001).

In the case of a low level of intra-cellular LDH, the cells are labelled with a fluorescent agent and the lysis is estimated by measuring the quantity of fluorescence released in the supernatant.

The specific lysis results are expressed as a percentage of lysis as a function of the antibody concentration. The EC₅₀ values (quantity of antibodies inducing 50% of maximum lysis) as well as the Eₘₐₓ values (percentage of maximum lysis) are calculated using PRISM software.

### Example 3

### Preparation of Cell Lines Expressing the BDCA-2 Protein

The complementary DNA of BDCA-2 is amplified by PCR and cloned in the expression vector pcDNA3.1(−) at the HindIII/BamHI restriction sites. The pcDNA vector containing BDCA-2 is transfected by nucleofection or Fugene HD on the Jurkat cell line. 48 hours after transfection, the expression of BDCA-2 by the transfected cells is monitored by flow cytometry, using an anti-BDCA-2 antibody coupled to a fluorophore. The BDCA-2+ cells are isolated using successive screenings carried out by flow cytometry with a sorter (Altra, Becton Dickson) using an anti BDCA-2 antibody (Myltenyi). The screening is repeated until 100% positive cells expressing BDCA-2 are obtained.

### Example 4

### Cytotoxicity

Depletion of Plasmacytoid DC Cells in PBMC

The PBMCs are isolated from the peripheral blood on a Ficoll gradient. The technique consists of incubating the PBMCs in the presence of a rabbit anti BDCA-2 polyclonal antibody or an irrelevant rabbit polyclonal antibody. After incubation for 16 hours, the depletion of the plasmacytoid DC cells induced by the anti BDCA-2 antibodies is measured by flow cytometry on the basis of ILT7/CD123 double labelling.

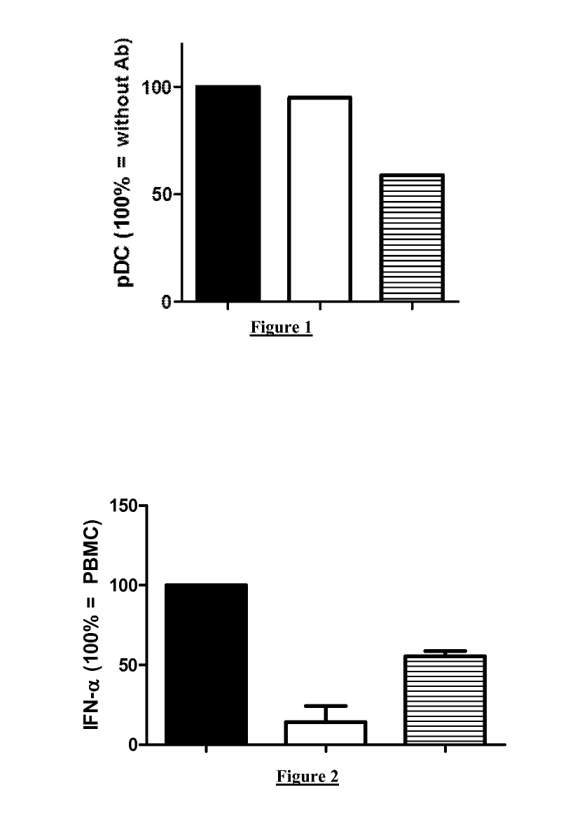

The results are shown in FIG. 1.

These results show a reduction in the number of plasmacytoid DC cells of approximately 40% in the presence of anti-BDCA-2 antibodies in comparison with the control antibody.

This result makes it possible to conclude that an anti-BDCA-2 antibody, in the presence of effector cells, can induce a depletion of the plasmacytoid DC cells.

Reduction in the Level of IFNα Associated with the Depletion of the Plasmacytoid DC Cells

The PBMCs are isolated from the peripheral blood on a Ficoll gradient. The technique consists of depleting plasmacytoid DC cells of the PBMCs using the Myltenyi anti plasmacytoid DC cells kit. After activation by CpG for 16 h at 37° C., the level of IFN-α is measured by flow cytometry and compared with the control PBMC containing the plasmacytoid DC cells.

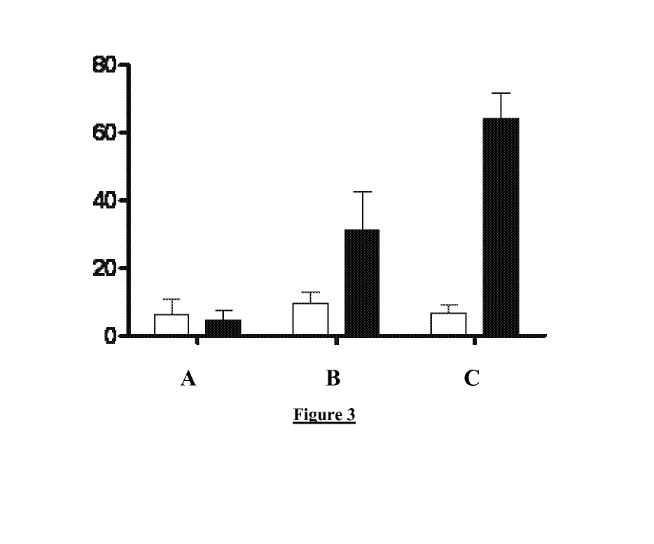

The results are shown in FIG. 2.

The results indicate a reduction in the secretion of IFN-α after approximately 85% depletion of the plasmacytoid DC cells, making it possible to conclude that the depletion of the plasmacytoid DCs by an anti-BDCA-2 can lead to a reduction in the secretion of IFN-α. In this experiment, the plasmacytoid DC cells alone are used as positive cells for the secretion of IFN-α.

ADCC

The killer cells (NK cells) are purified beforehand by the negative depletion technique developed by the company Miltenyi (Miltenyi Biotec—NK cell isolation kit human ref 130-092-657), from peripheral blood of healthy donors. The ADCC technique consists of incubating the NK cells with the target cells of the Jurkat line transfected with the BDCA-2 receptor, in the presence of different concentrations of a rabbit anti-BDCA-2 polyclonal antibody or an irrelevant rabbit polyclonal antibody. After incubation for 16 hours, the cytotoxic activity induced by the anti-BDCA-2 antibody is measured by colorimetry by assaying, in the supernatants, an intracellular enzyme called lactate dehydrogenase (LDH) released by the lysed target cells (Roche Diagnostics—Cytotoxicity Detection Kit LDH ref 11644793001).

The results are presented in FIG. 3 and show a cytotoxic activity of the rabbit anti-BDCA-2 polyclonal antibody on the Jurkat-BDCA-2 cells, in a dose-dependent manner which could reach more than 60% lysis of the target cell.

These results therefore indicate that by targeting the BDCA-2 antigen expressed at the surface of a cell, it is possible to lyse this cell using effectors such as NK cells. The anti-BDCA-2 antibody can support other cytotoxic activities such as the dependent phagocytosis of other effector cells such as macrophages or neutrophiles.

1-10. (canceled)

11. A method of inhibiting or treating pathologies involving activation of the plasmacytoid dendritic cells, comprising administering an effective amount of monoclonal or polyclonal antibody, or fragment thereof, directed against BDCA-2 protein to a subject in need thereof, wherein, said pathologies are haematopoietic tumours of phenotype CD4+, CD56+.

12. The method according to claim 11, wherein the tumours are haematopoietic tumours selected from the group consisting of:
lymphoproliferative syndromes and myeloma selected from the group consisting of Waldenström's disease, chronic lymphoid leukaemia, multiple myeloma, and primitive amyloidosis;
lymphomas selected from the group consisting of follicular lymphoma, diffuse lymphoma, lymphoma of the marginal zone, and Hodgkin's disease;
aplasia, leukaemias and myelodysplasias selected from the group consisting of acute lymphoblastic leukaemia, acute myeloid leukaemia, myelodysplasia, and medullar aplasia; and
myeloproliferative syndromes selected from the group consisting of myelofibrosis, polycythaemia vera, essential thrombocythaemia, chronic myeloid and leukaemia.

13. The method according claim 11, wherein said monoclonal or polyclonal antibody, or fragment thereof, is selected from the group consisting of: a murine antibody, a chimeric antibody, a humanized antibody, a human antibody, and fragments thereof.

14. The method according to claim 13, wherein said monoclonal or polyclonal antibody, or fragment thereof, is a chimeric antibody, or fragment thereof.

15. The method according to claim 14, wherein said chimeric antibody is a murine/human chimeric antibody or a human/macaque chimeric antibody.

16. The method according to claim 13, wherein said monoclonal or polyclonal antibody, or fragment thereof, is a humanized antibody, or fragment thereof.

17. The method according to claim 13, where said monoclonal or polyclonal antibody, or fragment thereof, is a monoclonal human antibody, or fragment thereof.

18. The method according to claim 11, where said monoclonal or polyclonal antibody, or fragment thereof, has a fucosylation level of less than 60% of the glycosylated forms.

19. The method according to claim 11, wherein said monoclonal or polyclonal antibody, or fragment thereof, is coupled to a bioactive molecule selected from the group consisting of:
radio-isotopes;
non-radioactive metals;
toxins selected from the group consisting of ricin, abrin, and diphtheria toxin;
the nucleic acids chosen from anti-sense RNAs; and
cytotoxic agents selected from the group consisting of:
antifolates selected from the group consisting of methotrexate, pemetrexed and raltitrexed,
anti-purines selected from the group consisting of cladribine, fludarabine,
azathioprine, azathioprine, mercaptopurine, 5-fluorouracile capecitabine, cytarabine and gemcitabine,
inhibitors of topoisomerases I and II,
alkylating agents selected from the group consisting of chlormethine, cyclophosphamide, ifosfamide, carmustine, and fotemustine and related agents selected from the group consisting of mitomycin C, cisplatin, carboplatin, and oxaliplatin,
intercalating agents,
anthracycline selected from the group consisting of daunorubicin, doxorubicin and chlorydrate, epirubicin, idarubicin, and bleomycin,
taxanes,
tyrosine-kinase specific inhibitors imatinib and Erlotinib, and
enzymes selected from the group consisting of RNases, biotin, avidin and streptavidin.

20. The method according to claim 11, wherein said effect amount of said monoclonal or polyclonal antibody is administered as a combination product comprising said monoclonal or polyclonal antibody, or fragment thereof, and an antineoplastic agent, and said monoclonal or polyclonal antibody, or fragment thereof, and an antineoplastic agent are administered simultaneously, separately or spread over time, in tumour therapy, in the treatment of haematopoietic tumours of phenotype CD4+, CD56+.

21. The method of according to claim 11, wherein said fragment is selected from the group consisting of Fab, F(ab)′2, Fd, scFV, ScFv dimer, diabody, triabody and tetrabody.
